(19) United States
(12) Patent Application Publication
(10) Pub. No.: US 2017/0196473 A1
(43) Pub. Date: Jul. 13, 2017

(54) QUANTITATIVE HEART MONITORING AND DIAGNOSTICS

(71) Applicant: Heart Test Laboratories, Inc., Westlake, TX (US)

(72) Inventors: David Krubsack, Allen, TX (US); Mark Hilz, Argyle, TX (US)

(21) Appl. No.: 15/400,527

(22) Filed: Jan. 6, 2017

Related U.S. Application Data
(60) Provisional application No. 62/276,639, filed on Jan. 8, 2016.

(51) Int. Cl. A61B 5/04; A61B 5/044; A61B 5/0452; A61B 5/0408; A61B 5/00

(52) U.S. Cl. CPC A61B 5/04012; A61B 5/04085; A61B 5/7275; A61B 5/0452; A61B 5/044

(57) ABSTRACT

Described are systems, devices, and methods for deriving quantitative metrics of heart condition and function from one or more electrocardiograms or time-frequency maps derived therefrom. In various embodiments, repolarization indices for the left and right ventricles are determined from the T wave in respective electrocardiograms. An overall heart-health index may be determined based on a comparison between the left and right ventricular repolarization indices.

### CROSS-REFERENCE TO RELATED APPLICATION

This application claims priority to and the benefit of, and incorporates herein by reference in its entirety, U.S. Provisional Patent Application No. 62/276,639, filed on Jan. 8, 2016.

### TECHNICAL FIELD

The present disclosure relates generally to heart testing, and more particularly to systems, devices, and methods for quantifying heart condition.

### BACKGROUND

Heart testing for coronary heart disease, myocardial ischemia, and other abnormal heart conditions is routinely performed using an electrocardiogram (ECG), which measures, via electrodes placed on the patient's skin, electrical potentials reflecting the electrical activity of the heart. In the heart, the heart's electrical system controls timing of the heartbeat by sending an electrical signal through the cells of the heart. The heart includes conducting cells for carrying the heart's electrical signal, and muscle cells that contract the chambers of the heart as triggered by the heart's electrical signal. The electrical signal starts in a group of cells at the top of the heart called the sinoatrial (SA) node. The signal then travels down through the heart, conducting cell to conducting cell, triggering first the two upper heart chambers (atria) and then the two lower heart chambers (ventricles). The left ventricle receives oxygenated blood, via the left atrium, from the lungs, and pumps the blood into the aorta, the main artery of the systemic circulation that supplies the body. The right ventricle receives oxygen-depleted blood, via the right atrium, from the body, and pumps it into the pulmonary artery for transport to the lungs.

Simplified, each heartbeat occurs by the SA node sending out an electrical impulse. The impulse travels through the atria, electrically polarizing the atria and causing them to contract. The atrioventricular (AV) node of the heart, located on the interatrial septum close to the tricuspid valve, sends an impulse into the ventricles of the heart, via the His-Purkinje system, causing the ventricles to depolarize and contract (or “pump”). Following the subsequent repolarization of the ventricles, the SA node sends another signal to the atria to contract, restarting the cycle. This pattern and variations therein indicative of disease are detectable in an ECG, and allow schooled medical personal to draw inferences about the heart's condition. However, not every developing abnormality is immediately visible in an ECG, and, consequently, many patients are misdiagnosed as healthy. Accordingly, there is a need for improved heart-disease diagnostics.

### BRIEF DESCRIPTION OF THE DRAWINGS

FIG. 1 is a block diagram illustrating an example system for quantifying heart condition in accordance with various embodiments.

FIG. 2 is an example electrocardiogram, including a T wave, in accordance with various embodiments.

FIGS. 3A and 3B are graphs of an example ECG for a normal heart and a scalogram resulting from its wavelet transform, respectively, in accordance with one embodiment.

FIGS. 3C and 3D are graphs of an example ECG for an abnormal heart and a scalogram resulting from its wavelet transform, respectively, in accordance with one embodiment.

FIG. 4 is a flow chart of methods for quantifying and assessing heart condition, in accordance with various embodiments.

FIG. 5 is a perspective view of an example heart test device in accordance with various embodiments.

FIG. 6 is an example user interface combining ECGs and scalograms with an indices report, in accordance with various embodiments.

FIG. 7 is a block diagram of an example computer system, as may serve as processing facility in accordance with various embodiments.

### DESCRIPTION

Described herein are systems, devices, and methods for deriving quantitative metrics of heart condition and function from one or more ECGs. ECGs are time-domain signals reflecting the electric potential of the heart throughout one or more cardiac cycles, and generally exhibit a plurality of distinct features that repeat for each cardiac cycle and correspond to different phases or events in the cycle. It has been found that the signal portion associated with the T wave, which represents the repolarization of the ventricles, is a particularly suitable indicator for heart condition. Accordingly, in various embodiments, “repolarization measures” are determined based on values of either the ECGs, or of time-frequency transforms of the ECGs (e.g., short-term Fourier transforms or wavelet transforms), taken at one or more points or ranges in time associated with the T wave. The repolarization measures may be averaged (e.g., across heart beats or leads) or adjusted (e.g., based on age or gender) to yield repolarization indices.

Multiple ECGs may be acquired for multiple respective leads, each lead corresponding to a different electrode or combination of electrodes. As the electrodes are placed on the patient at different locations, some of the leads measure signals primarily for the left ventricle of the heart and others measure signals primarily for the right ventricle of the heart. Since the left ventricle pumps blood through the systemic circulation, which is much more extensive than the pulmonary circulation (the latter being supplied with blood from the right ventricle), the electric potential, or energy, of the left ventricle is generally greater than that of the right ventricle. Consequently, the magnitude of signals associated with the left ventricle, and repolarization measures or indices derived therefrom, tend to be greater than signal magnitudes and repolarization measures or indices associated with the right ventricle—provided that the signals are rendered comparable by a suitable normalization that removes overall differences in signal strength unrelated to the heart's condition and function. If the reverse relation is observed and, e.g., a right ventricular repolarization measure or index is greater than a left ventricular repolarization measure or index, this is a strong indicator of an abnormality in heart function. Similarly, if the right ventricular repolarization measure or index becomes comparable to (even if it is still slightly smaller than) the left ventricular repolarization measure or index, the heart may be deemed suspect of an abnormality. In accordance with various embodiments, this relationship between heart condition or function and the relative sizes of signals and repolarization measures or indices associated with the left and right ventricles is used by determining a heart-health index based on a comparison of left and right ventricular repolarization measures or indices computed from suitably normalized signals. The comparison may involve computing the ratio, difference, or some other function of the left and right ventricular repolarization measures or indices. The heart-health index may be displayed to a medical professional or compared against a threshold to render an automatic diagnosis of heart health.

The foregoing will be more readily understood from the following more detailed description of various embodiments and the accompanying drawings.

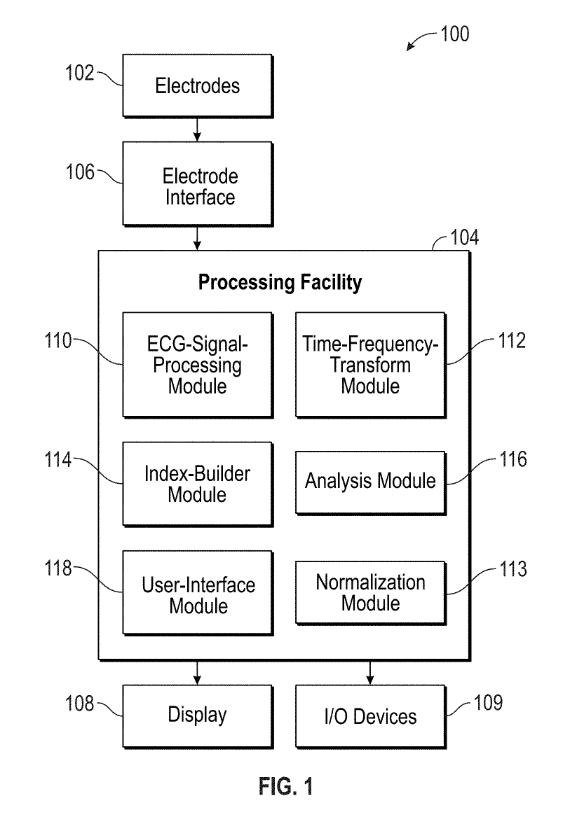

FIG. 1 illustrates, in block-diagram form, various functional components of an example system for quantifying heart condition in accordance with various embodiments. The system 100 includes one or more electrodes 102 for acquiring ECG signals (e.g., 10 electrodes for a traditional 12-lead ECG), a processing facility 104 for processing the ECG signals, e.g., to transform them into time-frequency maps and/or obtain repolarization measures and heart health indices, and an electrode interface 106 connecting the electrodes 102 to the processing facility 104. The electrode interface 106 includes circuitry that outputs electrical signals suitable as input to the processing facility 104, e.g., by digitally sampling analog input signals. The system 100 further includes a display device 108 for outputting the ECG test results (including, e.g., the ECGs, time-frequency maps, and/or repolarization measures and heart-health indices), and optionally other input/output devices 109, such as a keyboard and mouse and/or a printer, for instance. The display device 108 may be a touchscreen doubling as a user-input device. The processing facility 104, electrode interface 106, display 108, and input/output devices 109 may be implemented as a single, stand-alone device implementing all computational functionality for ECG signal processing and presentation. Alternatively, they may be provided by the combination of multiple communicatively coupled devices. For example, an ECG test device with limited functionality for recording and/or processing ECG signals received from one or more electrodes 102 via an electrode interface 106 of the device may outsource certain computationally intense processing tasks to other computers with which it is communicatively coupled via a wired or wireless network. Thus, the functionality of the processing facility 104 may be distributed between multiple computational devices that communicate with each other. Whether provided in a single device or distributed, the processing facility 104 may be implemented with dedicated, special-purpose circuitry (such as, e.g., a digital signal processor (DSP), field-programmable gate array (FPGA), analog circuitry, or other), a suitably programmed general-purpose computer (including at least one processor and associated memory), or a combination of both.

The processing facility 104 may include various functionally distinct modules, such as an ECG-signal-processing module 110 that prepares the (e.g., digitally sampled) electrical potentials for display (e.g., by filtering, smoothing, scaling, etc.); optionally a time-frequency transform module 112 that converts each ECG signal into a two-dimensional time-frequency map; a normalization module 113 that normalizes the ECGs or time-frequency maps to render them comparable; an index-builder module 114 that determines repolarization measures from the ECGs or time-frequency maps derived therefrom (which may involve, e.g., identifying delimiters between successive cardiac cycles, determining certain features (such as the QRS complex, RS segment, T wave, and other segments) within the ECGs, selecting points in time within the T wave, etc.) and computes a heart-health index; optionally an analysis module 116 that generates a diagnosis based on the heart-health index or the individual repolarization measures (e.g., by comparison against stored thresholds); and a user-interface 118 module that generates graphic representations of the data provided by the other modules and assembles them into a screen display.

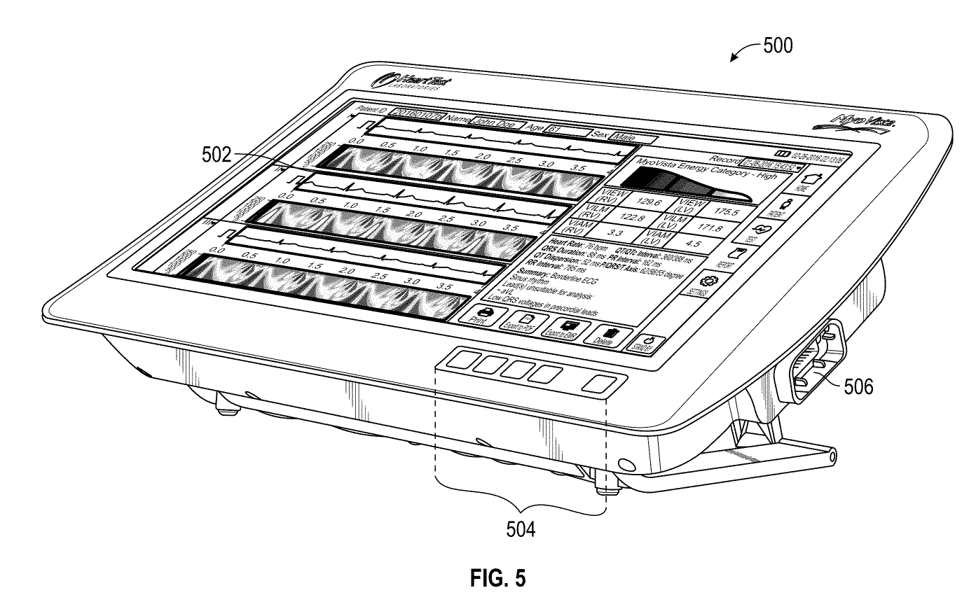

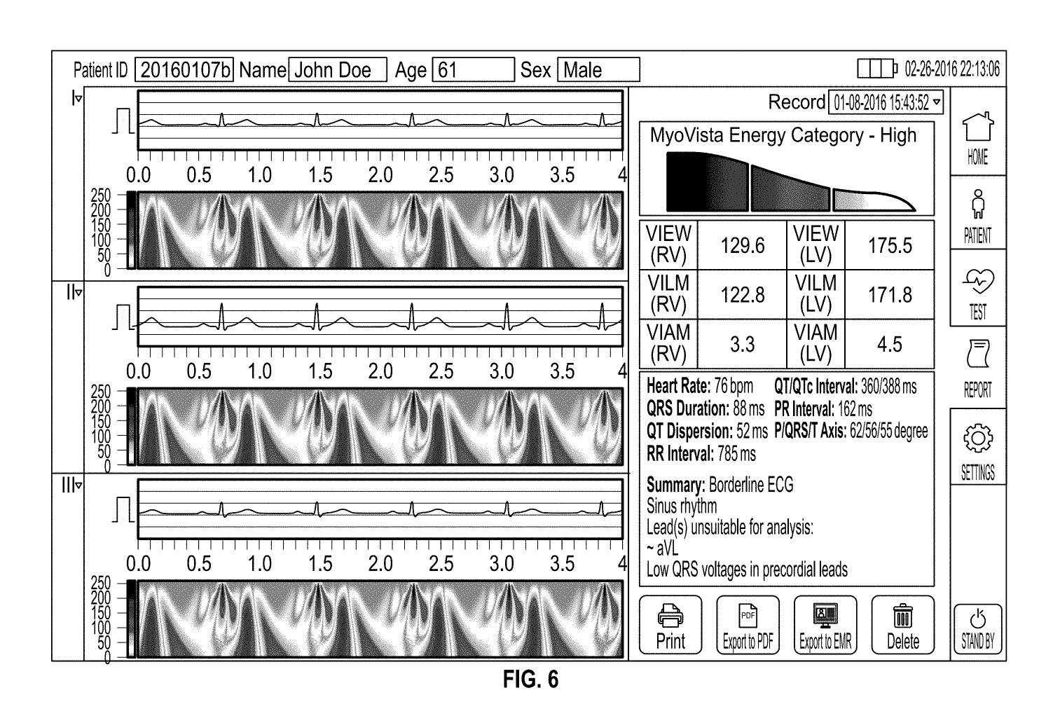

The ECG-signal-processing module 110 may be a conventional processing module as used in commercially available heart monitors and/or as capable of straightforward implementation by one of ordinary skill in the art. The time-frequency transform module 112, normalization module 113, index-builder module 114, and analysis module 116 implement algorithms explained in detail further below, and can likewise be readily implemented by one of ordinary skill in the art given the benefit of the present disclosure. The user-interface module 118 may implement routine functionality for generating user-interface screens in accordance with programmatic specifications; example output generated by this module 118 is shown in FIGS. 5 and 6 below. As will be readily appreciated, the depicted modules reflect merely one among several different possibilities for organizing the overall computational functionality of the processing facility 104. The modules may, of course, be further partitioned, combined, or altered to distribute the functionality differently. For example, the normalization functionality of module 113 may, instead, be integrated into the ECG-signal-processing and time-frequency transform modules 110, 112, or into the index-builder module 114. The various modules may be implemented as hardware modules, software modules executed by a general-purpose processor, or a combination of both. For example, it is conceivable to implement the time-frequency transform module 112, which generally involves the same operations for each incoming ECG signal, with special-purpose circuitry to optimize performance, while implementing the index-builder module 114 and the analysis module 116 in software to provide flexibility for adjusting parameters and algorithms, e.g., in response to new medical data.

While the quantification of heart function in accordance herewith is, in general, not limited to any particular number of electrodes, the system 100 includes, in various embodiments, ten electrodes 102 to facilitate obtaining a standard twelve-lead ECG, as is routinely used in the medical arts. In accordance with the standard configuration, four of the ten electrodes (conventionally labeled LA, RA, LL, RL) are placed on the patient's left and right arms and legs; two electrodes (labeled V1 and V2) are placed between the fourth and fifth ribs on the left and right side of the sternum; a further, single electrode (labeled V3) is placed between V2 and V4 on the fourth intercostal space; one electrode (labeled V4) is placed between the fifth and sixth ribs at the mid-clavicular line (the imaginary reference line that extends down from the middle of the clavicle), and, in line therewith, another electrode (labeled V5) is positioned in the anterior axillary line (the imaginary reference line running southward from the point where the collarbone and arm meet), and the tenth electrode (labeled V6) is placed on the same horizontal line as these two, but oriented along the mid-axillary line (the imaginary reference point straight down from the patient's armpit). The electric potentials measured by electrodes V1 through V6 correspond to six of the twelve standard leads; the remaining six leads correspond to the following combinations of the signals measured with the individual electrodes: I=LA−RA; II=LL−RA; III=LL−LA; aVR=RA−½ (LA+LL); aVL=LA−½ (RA+LL); and aVF=LL−½(RA+LA). In accordance with various embodiments, left ventricular repolarization measures are determined based on ECGs for leads V4, V5, and V6, and right ventricular repolarization measures are based on ECGs for leads V1 and V2.

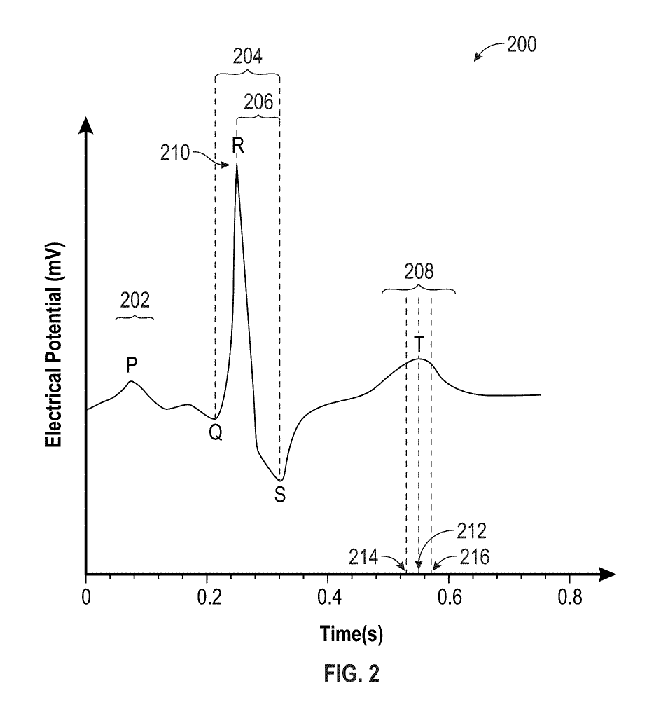

FIG. 2 schematically shows an example ECG 200 for a single cardiac cycle, illustrating the P wave 202, QRS complex 204 (which includes the RS segment 206), and T wave 208. As depicted, the electric potential usually reaches its maximum 210 at R during the QRS complex 204. In many instances, this renders the R wave suitable for normalizing the ECGs. However, the polarity of the signal may be inverted (such that the R peak has a negative value). Further, in some ECG signals, the S peak has a greater absolute value than the R peak. In fact, not every ECG unambiguously exhibits the features shown in the (rather typical) example ECG 200. This uncertainty can cause difficulty in attempts to normalize the signal based on a discrete feature of the ECG such as, e.g., the R peak. To circumvent this difficulty and provide for a more robust normalization, various embodiments base normalization, instead, on a signal maximum and minimum identified across a time range, such as the time interval encompassing at least the RS segment 206 (and thus including both the R and the S peak if they are, in fact, clearly represented in the signal), irrespective of the feature to which that maximum or minimum corresponds (if any). This normalization is more fully explained below.

FIG. 2 also illustrates certain points in time at which data is evaluated in accordance with various embodiments, such as the time 212 at which the T wave 208 assumes its maximum, and example “early” and “late” times 214, 216 bracketing the maximum of the T wave. In general, the early and late times 214, 216 may be anywhere on the rising edge and falling edge, respectively, of the T wave. In various embodiments, the early and late times 214, 216 are selected in the vicinity of the maximum in the sense that they are within ranges between the T wave maximum and points in time preceding and following the T wave maximum, respectively, at which the T wave assumes some specified fraction, e.g., half, of its maximum value. The early and late times may be specified in terms of fixed distances from the time 212 of the T wave peak. For example, they may be +/−0.01 seconds or +/−0.08 seconds from the peak time 212.

In accordance with various embodiments, repolarization measures are computed from values of the ECGs taken at one or more points or ranges in time associated with the T wave. For example, a repolarization measure may be based on the peak value of the T wave, on the signal values at early and late times defined somewhere on the rising edge and falling edge of the T wave, respectively, or on some combination (e.g., weighted average) of these values. In some embodiments, a repolarization measure is computed as the difference between the maximum and minimum values within a specified time interval surrounding the T wave peak (e.g., the interval between points in time 214, 216), which is typically, but not necessarily, the difference between the peak value and the smaller one of the values at the boundaries of the time interval (e.g., the value at time point 214 or at time point 216). Beneficially, defining the repolarization measure in this manner obviates the need to determine where the baseline of the ECG signal (corresponding to a zero signal value) is, which can sometimes be ambiguous, and furthermore provides a straightforward and simple prescription for computing the repolarization measure even if the T wave deviates from a smooth curve due to, e.g., noise (which can cause the extrema within the specified time interval to be at points other than the peak time and the boundaries of the interval). Instead of using instantaneous values for the repolarization measures, the signals may also be integrated over a narrow sliding window of fixed width, e.g., for the purposes of reducing the effect of noise in the ECGs. Further, in accordance with some embodiments, repolarization measures are computed from integrals over larger ranges in time, e.g., over the entire time period associated with the T wave. Also, instead of integrating over the original ECG signal representing an electrical potential, the integral may be formed over the squared signal to obtain a measure corresponding to the energy within a selected signal portion (e.g., the energy within the T wave).

In some embodiments, as described above, repolarization measures are computed directly from the time-domain ECGs. In alternative embodiments, described in the following, the measured ECGs are first transformed into two-dimensional time-frequency maps by a suitable mathematical transform (herein “time-frequency transform”), such as, for instance, short-term Fourier transform, (discrete or continuous) wavelet transform, or by application of a filter bank (of which wavelet transform is one example). The repolarization measures can then be determined from the time-frequency map, e.g., by selecting the maximum across the spanned frequency range at one or more points in time (e.g., the time where the T wave in the ECG peaks, or at early or late times within the ECG), or by integrating the map values, or squared values, over both frequency and time to obtain energy measures.

In some embodiments, the ECGs are transformed by wavelet transform. For a given continuous ECG signal x(t), the continuous wavelet transform is given by:

$$W ( a , b ) = \frac{1}{\sqrt{a}} {\int }_{- ∞}^{+ ∞} \overline{ψ ( \frac{t - b}{a} )} x ( t ) dt ,$$

where ψ is a selected wavelet, b corresponds to a shifted position in time and a to a scaling factor, and W(a, b) is the two-dimensional function of position in time and scale resulting from the transform, also called wavelet coefficients. Similarly, for a discretized ECG signal x(k) (where k is an integer), the continuous wavelet transform is given by:

$$W ( a , b ) = \frac{1}{\sqrt{a}} {\sum }_{k} x ( k ) ( {\int }_{- ∞}^{( k + 1 ) T} \overline{ψ ( \frac{t - b}{a} )} dt - {\int }_{- ∞}^{kT} \overline{ψ ( \frac{t - b}{a} )} dt ) ,$$

where T is the sampling period. The wavelet selected for processing may be, for example, a Mexican hat wavelet, Morlet wavelet, Meyer wavelet, Shannon wavelet, Spline wavelet, or other wavelet known to those of ordinary skill in the art. The result of the wavelet transform, W(a, b), is also referred to as a scalogram, which can provide more useful or intuitive visual insights into heart condition and function.

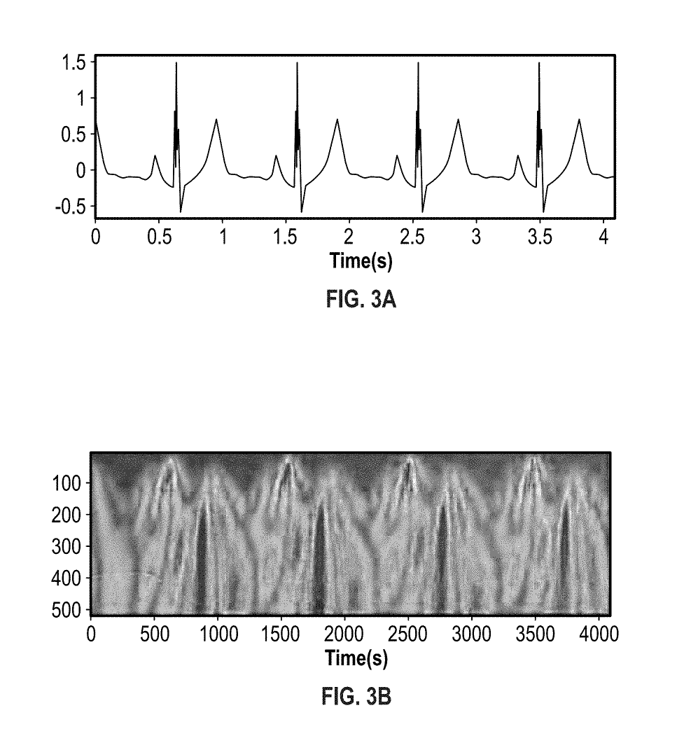

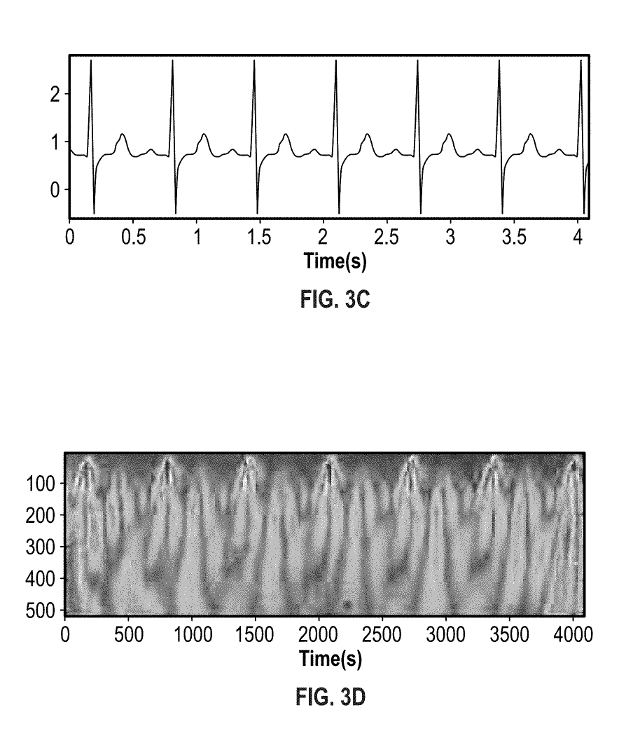

FIGS. 3A and 3B illustrate an example ECG for a normal heart and an unsigned scalogram resulting from its wavelet transform (followed by taking the absolute), respectively. In the scalogram, the position b corresponding to time is along the abscissa and the scale a (corresponding to frequency) along the ordinate, and the signal value W is encoded by color or intensity (e.g., gray-scale value). As can be seen, the various peaks of the normal ECG are reflected in relatively high intensity in the scalogram, allowing identification of the different ECG segments. For comparison, FIGS. 3C and 3D show an example ECG and associated scalogram, respectively, for an abnormal heart. Here, features that are prominent in the normal scalogram (e.g., the T wave) have rather low intensity. While this lower intensity generally tracks the lower values of the T wave in the ECG, it will be appreciated that the scalogram may provide better visual clues. Accordingly, the scalogram can aid a physician or other skilled clinician to assess heart functioning.

The time-frequency maps (such as, e.g., scalograms) generally include both positive and negative values. For an intuitive interpretation of the signal value of the time-frequency map as a measure of the electrical energy of the heart, however, the sign is not relevant (since, in a measure of the energy, the electrical potential is squared). Accordingly, in some embodiments, the absolute value of the signal value (or the square of the signal value) is taken at each time-frequency point, resulting in an unsigned time-frequency map. The unsigned time-frequency map may be advantageous, in particular, for display in a user interface (e.g., to a physician) since it avoids presenting information that is not of immediate, intuitively discernible clinical significance and is potentially distracting. On the other hand, since the signed time-frequency map contains generally more information than the unsigned time-frequency map, the computation of repolarization measures and indices may (but need not) be based on the signed map.

The time-frequency maps (optionally following normalization as explained further below) may be displayed to a physician for evaluation. Alternatively or additionally, they may be further analyzed, in accordance with various embodiments, to determine various quantitative indicators of heart condition and function. To that end, various measures of the electric activity of the heart can be obtained, e.g., by determining extrema (i.e., maximum and/or minimum values) across frequency of the (normalized) time-frequency map at certain points (or ranges) in time corresponding to distinctive features of the underlying ECGs, in particular, certain points (or ranges) in time associated with the T wave. Repolarization measures, which are associated with the T wave, include, for example, the maximum value at an early time within the T wave (REM), the maximum value at a late time within the T wave (RLM), or the maximum value at the peak of the T wave (RPM). Additional repolarization measures, e.g., including an integral over a time interval within the T wave, may also be defined and used to quantify heart condition.

To facilitate meaningful comparisons between ECGs obtained simultaneously for different leads and/or between time-frequency maps derived therefrom, the ECGs and/or time-frequency maps may be normalized. Normalization may involve scaling and/or shifting signal values in the ECG or time-frequency map to map the range of signal values (across the entire ECG or time-frequency map or at least a portion thereof, as explained below) to a specified numerical range (hereinafter “target range”), e.g., 0 to 255 or -128 to +127 (as are convenient ranges for binary representations, and can, for the display of time-frequency maps, be straightforwardly mapped onto color or gray-scale values). Using a particular normalization and the associated target range consistently not only across leads, but also across measurements taken at different times and/or even for different patients may also serve to improve comparability of data over time and across the patient population, as it eliminates or at least reduces overall signal-level variations, which are often not attributable to different heart conditions, allowing physicians to focus on the clinically relevant relative signal levels within an ECG or a time-frequency map.

The normalization may be based on a regional maximum and minimum defined as the maximum and minimum of the ECG within a selected time interval, or of the time-frequency map across frequency and across time within a selected time interval, and may then be applied to a second selected interval that may or may not be the same as the first selected interval. The maximum and minimum of the ECG across that second time interval, or of the time-frequency map across frequency and across time within that second selected time interval, are hereinafter called the absolute maximum and minimum, and they may, but need not, coincide with the regional maximum and minimum. The first selected interval is typically, but not required to be, coextensive with or shorter than the second selected interval. In some embodiments, the regional maximum and minimum for the ECG or time-frequency map derived therefrom are determined across the entire measurement time of the ECG, and the normalization is applied over that same range (such that the first and second selected intervals are equal). In other embodiments, the regional maximum and minimum are identified within a portion of the ECG or time-frequency map that is limited in its time dimension, e.g., to an integer number of heartbeats (e.g., disregarding partial heartbeats) or only a single heartbeat. An ECG or time-frequency map encompassing multiple heartbeats may, for instance, be broken up into portions corresponding to individual heartbeats, and each portion may be normalized separately (potentially resulting in some discontinuity of the signal values in the normalized ECG or time-frequency map); in this case, first and second selected intervals are likewise equal to each other. Normalization may even be based on a time interval encompassing only part of a heartbeat, selected to likely (but not certainly) include the absolute maximum and minimum. For instance, in some embodiments, regional maximum and minimum are determined within a portion of an ECG or time-frequency map that encompasses at least the RS segment. Note, however, that it is possible for, e.g., the T wave maximum to exceed the maximum in the QRS complex. In cases where the absolute maximum and minimum of the ECG or time-frequency map lie outside the portion of the map across which the regional maximum and minimum are determined, the normalization will result in signal values exceeding the target range.

Normalization may be applied according to the following equation:

$$n = ( d - {d}_{min} ) * \frac{( {n}_{max} - {n}_{min} )}{( {d}_{max} - {d}_{min} )} + {n}_{min} ,$$

where

n is the normalized data point;

nₘᵢₙ is the normalized target-range minimum;

nₘₐₓ is the normalized target-range maximum;

d is the data point to be normalized;

dₘᵢₙ is the regional minimum; and

dₘₐₓ is the regional maximum.

For example, to map onto the target range from 0 to 255, nₘᵢₙ is 0 and nₘₐₓ is 255; in effect, this normalization shifts the ECG or time-frequency map to a minimum equal to zero and thereafter scales the shifted ECG or map based on its shifted regional maximum. More generally, the normalization shifts the ECG or time-frequency map to a minimum equal to nₘᵢₙ and then scales the values of the shifted ECG or time-frequency map (taken relative to the minimum value) by the ratio of the difference between maximum and minimum of the target range to the difference between the regional maximum and minimum.

Normalization can be applied to signed as well as unsigned time-frequency maps. As will be appreciated, the result of the normalization will vary depending on whether the underlying time-frequency map is signed or unsigned. For example, when mapping a signed time-frequency map with a positive R peak and a negative S peak onto the target range from 0 to 255, several of the frequencies at the point in time corresponding to the S peak will map to or near zero. However, when the normalization is applied to the absolute value of the otherwise same time-frequency map, some frequencies at points in time between R and S will now map to or near zero whereas several of the frequencies at the point in time corresponding to the S peak will map onto a relatively larger positive number within the target range.

From the repolarization measures determined in the ECGs or time-frequency maps, one or more repolarization indices may be derived, e.g., by averaging or by adjustment based on information external to the ECG or time-frequency map. For example, if the repolarization measures are obtained based on ECGs covering multiple cardiac cycles, the individually determined maxima may be averaged over these cycles. Further, the various repolarization measures can generally be derived separately from ECGs or time-frequency maps for different respective leads, and repolarization measures of the same type (e.g., the REMs) may be averaged across multiple leads. In particular, ventricular repolarization indices may be derived by averaging only across leads associated with the same (i.e., left or right) ventricle. For example, a ventricular index early measure for the right ventricle (VIEM_RV) may be calculated by (e.g., arithmetically) averaging over the REMs of leads V1 and V2, a ventricular index late measure for the right ventricle (VILM_RV) may be calculated by averaging over the RLMs of leads V1 and V2, and a ventricular index peak measure for the right ventricle (VIPM_RV) may be calculated by averaging over the RPMs of leads V1 and V2. Similarly, VIEM, VILM, and/or VIPM for the left ventricle (VIEM_LV, VILM_LV, and VIPM_RV) may be calculated by averaging over the REMs, RLMs, and RPMs, respectively, of leads V4, V5, and V6. In certain embodiments, further indices are derived from the preceding ones. For instance, a ventricular index average measure for the right ventricle (VIAM_RV) may be calculated as the sum of VIEM_RV and VILM_RV, divided by the heart rate (measured in beats per minute). Similarly, a ventricular index average measure for the left ventricle (VIAM_RV) may be calculated as the sum of VIEM_LV and VILM_LV, divided by the heart rate.

Further, while the repolarization measures are generally indicators of how well the heart functions, they can also be affected by age and gender, independently of any abnormal heart condition. To eliminate or at least reduce differences that do not result from heart abnormalities, the repolarization measures may be adjusted, when computing repolarization indices, with suitable age- and/or gender-dependent factors. In one embodiment, the adjustment distinguishes merely between male and female patients, using an adjustment factor of 1 for males (i.e., keeping the measures as is) and an adjustment factor smaller than one (e.g., 1/1.24) for females. In some embodiments, further refinements are made to distinguish between patients up to forty years old and patients older than forty years. For example, for females older than forty years, the adjustment factor may be decreased to 1/1.26. Other age-based classifications and adjustment factors may be implemented as well.

In accordance with various embodiments, as described above, left and right ventricular repolarization indices are computed from the electrocardiograms, or time-frequency transforms thereof, to provide quantitative indicators of the condition and function of the left and right ventricles. Further, in some embodiments, an overall heart-health index is determined based on a comparison (broadly understood) of the left and right ventricular indices in view of the expectation that, for a healthy heart, the left ventricular repolarization index should exceed the right ventricular repolarization index. (As will be appreciated, such comparisons are meaningful only if the repolarization measures computed for the left and right ventricles are of the same type and normalized.) In some embodiments, the heart-health index is the ratio of the left ventricular repolarization index to the right ventricular repolarization index (or some function of the ratio). This ratio can be compared against a threshold of, e.g., 1. A ratio smaller than 1 may be cause for a diagnosis of an abnormal heart. Alternatively, the heart-health index may be the difference between the left and right ventricular repolarization indices (or some function of the difference). If the left ventricular repolarization index minus the right ventricular repolarization index is smaller than 0, this is, again, indicative of an abnormal heart. More generally, the heart-health index may be some function (including, e.g., a piecewise defined function) of the left and right repolarization indices that, in some form or another, implies a comparison between the two. Note that thresholds need not necessarily be set to merely discriminate between situations where the left ventricular repolarization index is greater vs. smaller than the right ventricular repolarization index. Rather, a suspect or abnormal heart condition may be found, e.g., if the left ventricular repolarization index is not greater than the right ventricular repolarization index by the expected amount.

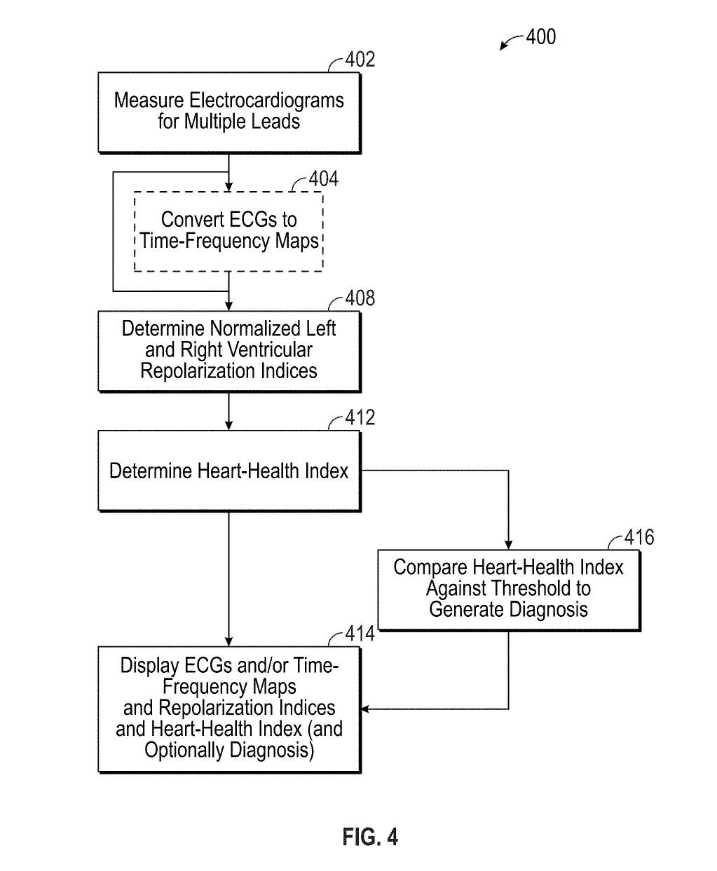

FIG. 4 is a flow chart of an example method 400 for quantifying and assessing heart condition, in accordance with various embodiments. The method 400 involves measuring multiple ECGs for respective leads that include one or more leads associated with the left ventricle and one or more leads associated with the right ventricle (action 402), using multiple electrodes placed on a patient. In some embodiments, ten electrodes are used to obtain twelve leads, as described above. The ECGs may, optionally, be converted by time-frequency transform (e.g., wavelet transform) into respective two-dimensional time-frequency maps (e.g., scalograms) (action 404). To quantify the heart condition, normalized left and right ventricular repolarization measures are then determined based on the individual ECGs and/or the corresponding time-frequency maps and, if applicable, averaged across leads associated with the same ventricle (and/or otherwise adjusted) to yield left and right ventricular repolarization indices (action 408). (In the absence of any averaging or adjustment, the left and right ventricular repolarization measures constitute the left and right ventricular repolarization indices.) An overall heart-health index is determined from the left and right ventricular repolarization measures (action 412). The ECGs and/or time-frequency maps and the repolarization indices and heart-health index derived therefrom may be displayed to a user (e.g., a physician) (action 414). In some embodiments, the heart-health index is automatically compared against a specified threshold to generate an automated qualitative diagnosis (e.g., “normal,” “abnormal,” or “suspect”), which may likewise be displayed (e.g., symbolically via suitable icons) (action 416).

Referring now to FIG. 5, an example heart test device 500 is shown in perspective view. The depicted device takes the form of a tablet computer 500 including a touchscreen display 502 as well as a control panel 504 with physical buttons (e.g., to power the tablet 500 on/off). In some embodiments, as shown, the display 502 presents a multi-tab user interface (including, e.g., patient, test, and report screens). Some of the tabs (shown along the right edge of the display 502) may be duplicated by the physical buttons of the control panel 504, allowing an operator to navigate between different screens and associated device functions in different ways. On the touch-screen interface 502, e.g., in a report screen, the measured ECGs and the time-frequency maps (or subsets thereof), and/or a report of the repolarization measures/indices, heart-health index, and/or a symbolic indicator of the overall diagnosis may be displayed. Electrodes for acquiring the ECG signals may be hooked up to the tablet computer 500 via a suitable connector 506 (e.g., a DB15 connector). The tablet 500 contains a general-purpose processor and volatile as well as non-volatile memory storing instructions for implementing the functional processing modules 110, 112, 113 114, 116, 118. Of course, in various alternative embodiments, the heart test device may take different form factors, such as that of a desktop computer, laptop computer, or smartphone (to name just a few), each with a suitable electrode interface, which may include custom circuitry for converting the electrode signals into digital signals suitable for further processing with software. Furthermore, an electrocardiography system providing the functionality described herein need not necessarily be implemented in a single device, but can be provided by multiple devices used in combination, e.g., a conventional ECG monitor connected to a general-purpose computer running software to implement the processing functionality described herein.

FIG. 6 illustrates an example report screen in accordance with various embodiments. As shown, the report screen may be partitioned into multiple screen portions arranged in an intuitive manner so as to allow the viewer to quickly locate the desired information. At the top of the screen, patient information, such as a unique patient identifier and the patient's name, as well as patient-specific parameters affecting the interpretation of the ECGs, such as age and gender, may be displayed, along with a record identifier composed of, e.g., a date and time stamp for the test. In a left panel, ECGs and time-frequency maps for one or more leads (e.g., as depicted, leads I, II, and III) may be displayed, e.g., in a vertical arrangement. In accordance with various embodiments, the signal value of the time-frequency map (e.g., the electrical potential or voltage that is plotted as a function of time and frequency) is encoded in a color scale (or, alternatively, as shown in the black-and-white drawings, in a grey scale). While the signal value itself, as resulting from the time-frequency (e.g., short-time Fourier or wavelet) transform applied to the ECG, may be a signed value (generally resulting in both positive and negative values across the map), the color-coded depicted value may be unsigned, as obtained from a signed value by computing the absolute value. Using unsigned signal values in the color-coded maps serves to represent the energy level of the time-dependent frequency content, independent of the phase of those frequencies, thus allowing the energy of either positive or negative phase to appear at the same point (along frequency) on the time-frequency map. The report screen further shows, in the right panel, various left and right ventricular repolarization indices (VIEM, VILM, VIAM, defined above) computed from the time-frequency maps. A physician may compare the values of these indices between left and right ventricles (indicated by “RV” and “LV”). Further, in some embodiments, a heart health index computed from the left and right ventricular indices (e.g., by subtracting the left from respective right indices) may be displayed (not shown in FIG. 6). Whether displayed or not, the heart health index may serve to automatically assess heart function, e.g., to categorize it as normal, abnormal, or suspect. This overall diagnosis may be symbolically represented. For example, in the depicted embodiment, a three-part waveform may be shown fully in color in the case of normal heart function, whereas suspect or abnormal function may be indicated by graying out one or two of the segments. Certain embodiments are described herein as including a number of logic components or modules. Modules may constitute either software modules (e.g., code embodied on a non-transitory machine-readable medium) or hardware-implemented modules. A hardware-implemented module is tangible unit capable of performing certain operations and may be configured or arranged in a certain manner. In example embodiments, one or more computer systems (e.g., a standalone, client or server computer system) or one or more processors may be configured by software (e.g., an application or application portion) as a hardware-implemented module that operates to perform certain operations as described herein.

In various embodiments, a hardware-implemented module may be implemented mechanically or electronically. For example, a hardware-implemented module may comprise dedicated circuitry or logic that is permanently configured (e.g., as a special-purpose processor, such as a field programmable gate array (FPGA) or an application-specific integrated circuit (ASIC)) to perform certain operations. A hardware-implemented module may also comprise programmable logic or circuitry (e.g., as encompassed within a general-purpose processor or other programmable processor) that is temporarily configured by software to perform certain operations. It will be appreciated that the decision to implement a hardware-implemented module mechanically, in dedicated and permanently configured circuitry, or in temporarily configured circuitry (e.g., configured by software) may be driven by cost and time considerations.

Accordingly, the term “hardware-implemented module” should be understood to encompass a tangible entity, be that an entity that is physically constructed, permanently configured (e.g., hardwired) or temporarily or transitorily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure a processor, for example, to constitute a particular hardware-implemented module at one instance of time and to constitute a different hardware-implemented module at a different instance of time.

Hardware-implemented modules can provide information to, and receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and store the output of that operation in a memory device to which it is communicatively coupled. A further hardware-implemented module may then, at a later time, access the memory device to retrieve and process the stored output. Hardware-implemented modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., Application Program Interfaces (APIs).)

Example embodiments may be implemented in digital electronic circuitry, or in computer hardware, firmware, software, or in combinations of them. Example embodiments may be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry, e.g., a field programmable gate array (FPGA) or an application-specific integrated circuit (ASIC).

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine) and software architectures that may be deployed, in various example embodiments.

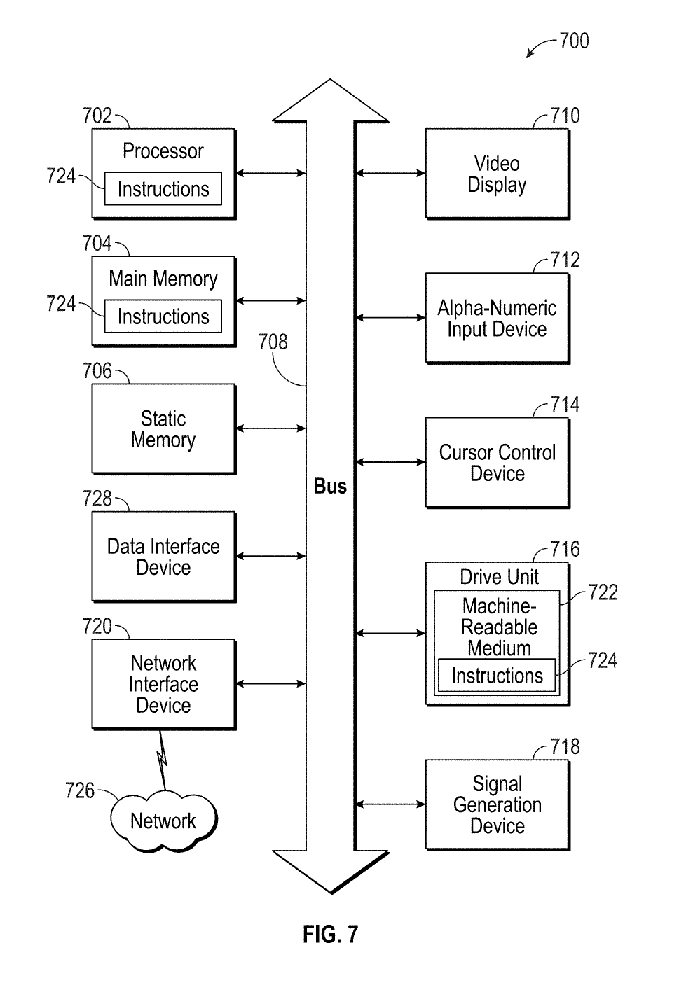

FIG. 7 is a block diagram of a machine in the example form of a computer system 700 within which instructions for causing the machine to perform any one or more of the methodologies discussed herein may be executed. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. While only a single machine is illustrated, the term “machine” shall also be taken to include any collection of machines that individually or jointly execute a set (or multiple sets) of instructions to perform any one or more of the methodologies discussed herein. The example computer system 700 includes a processor 702 (e.g., a central processing unit (CPU), a graphics processing unit (GPU) or both), a main memory 704 and a static memory 706, which communicate with each other via a bus 708. The computer system 700 may further include a video display unit 710 (e.g., a liquid crystal display (LCD) or a cathode ray tube (CRT)). The computer system 700 also includes an alphanumeric input device 712 (e.g., a keyboard), a user interface (UI) navigation device 714 (e.g., a mouse), a disk drive unit 716, a signal generation device 718 (e.g., a speaker), a network interface device 720, and a data interface device 728 (such as, e.g., an electrode interface 106).

The disk drive unit 716 includes a machine-readable medium 722 storing one or more sets of instructions and data structures (e.g., software) 724 embodying or utilized by any one or more of the methodologies or functions described herein. The instructions 724 may also reside, completely or at least partially, within the main memory 704 and/or within the processor 702 during execution thereof by the computer system 700, the main memory 704 and the processor 702 also constituting machine-readable media.

While the machine-readable medium 722 is shown in an example embodiment to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding, or carrying instructions for execution by the machine and that cause the machine to perform any one or more of the methodologies of the present invention, or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including by way of example semiconductor memory devices, e.g., Erasable Programmable Read-Only Memory (EPROM), Electrically Erasable Programmable Read-Only Memory (EEPROM), and flash memory devices; magnetic disks such as internal hard disks and removable disks; magneto-optical disks; and CD-ROM and DVD-ROM disks.

Although the invention has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense.

1. A method comprising:
using a plurality of electrodes placed on a patient, measuring a plurality of electrocardiograms for a plurality of respective leads, at least one of the leads being associated with a left ventricle and at least one of the leads being associated with a right ventricle, each electrocardiogram comprising at least one T wave;
using a computer processor,
computing a left ventricular repolarization index based on at least one electrocardiogram for the at least one lead associated with the left ventricle and computing a right ventricular repolarization index based on at least one electrocardiogram for the at least one lead associated with the right ventricle, the left and right ventricular repolarization indices being based on values of the respective electrocardiograms, or time-frequency transforms thereof, taken at one or more points or ranges in time associated with the T wave; and
computing a heart-health index based on a comparison of the left and right ventricular repolarization indices.

2. The method of claim 1, wherein the left and right ventricular repolarization indices are computed directly from respective time-domain electrocardiograms.

3. The method of claim 2, wherein left and right ventricular indices are computed from repolarization measures each corresponding to a difference between a maximum value and a minimum value of the respective electrocardiogram within a time interval surrounding a peak of the T wave.

4. The method of claim 2, further comprising, prior to computing the left and right ventricular repolarization indices, normalizing the electrocardiograms based at least in part on a difference between a maximum and a minimum identified in the respective electrocardiogram across a time interval encompassing an RS segment.

5. The method of claim 4, wherein normalizing the electrocardiograms comprises shifting each electrocardiogram to a minimum equal to zero and thereafter scaling the respective electrocardiogram based on the maximum.

6. The method of claim 1, wherein the left and right ventricular repolarization indices are computed from time-frequency maps obtained by time-frequency transform from respective time-domain electrocardiograms.

7. The method of claim 6, further comprising, prior to computing the left and right ventricular repolarization indices, normalizing the time-frequency maps based at least in part on a difference between a maximum and a minimum identified in the respective time-frequency map across time in an interval encompassing an RS segment and across frequency.

8. The method of claim 7, wherein normalizing the time-frequency maps comprises shifting each time-frequency map to a minimum equal to zero and thereafter scaling the respective time-frequency map based on the maximum.

9. The method of claim 1, wherein the heart-health index is a function of the left ventricular repolarization index and the right ventricular repolarization index.

10. The method of claim 9, wherein the heart-health index comprises a ratio of the left ventricular repolarization index and the right ventricular repolarization index.

11. The method of claim 9, wherein the heart-health index comprises a difference between the left ventricular repolarization index and the right ventricular repolarization index.

12. The method of claim 1, wherein the left ventricular repolarization index is based on electrocardiograms for leads V4, V5, and V6, and the right ventricular repolarization index is based on electrocardiograms for leads V1 and V2.

13. The method of claim 1, further comprising displaying the heart-health index to a user.

14. The method of claim 1, further comprising generating a diagnosis by comparing the heart-health index against a specified threshold.

15. A system comprising:
a plurality of electrodes and associated circuitry for measuring a plurality of electrocardiograms for a plurality of respective leads, at least one of the leads being associated with a left ventricle and at least one of the leads being associated with a right ventricle, each electrocardiogram comprising at least one T wave; and
a processing facility communicatively coupled to the circuitry and configured to
compute a left ventricular repolarization index based on at least one electrocardiogram for the at least one lead associated with the left ventricle and computing a right ventricular repolarization index based on at least one electrocardiogram for the at least one lead associated with the right ventricle, the left and right ventricular repolarization indices being based on values of the respective electrocardiograms, or time-frequency transforms thereof, taken at one or more points or ranges in time associated with the T wave; and
compute a heart-health index based on a comparison of the left and right ventricular repolarization indices.

16. The system of claim 15, further comprising a screen for displaying the heart-health index to a user.

17. A tangible machine-readable medium storing processor-executable instructions for processing a plurality of electrocardiograms for a plurality of respective leads, at least one of the leads being associated with a left ventricle and at least one of the leads being associated with a right ventricle, each electrocardiogram comprising at least one T wave, the instructions, when executed by one or more processor, causing the processor to perform operations comprising:
compute a left ventricular repolarization index based on at least one electrocardiogram for the at least one lead associated with the left ventricle and computing a right ventricular repolarization index based on at least one electrocardiogram for the at least one lead associated with the right ventricle, the left and right ventricular repolarization indices being based on values of the respective electrocardiograms, or time-frequency transforms thereof, taken at one or more points or ranges in time associated with the T wave; and
compute a heart-health index based on a comparison of the left and right ventricular repolarization indices.

18. The tangible machine-readable medium of claim 17, wherein the left ventricular repolarization index is based on electrocardiograms for leads V4, V5, and V6, and the right ventricular repolarization index is based on electrocardiograms for leads V1 and V2.

19. The tangible machine-readable medium of claim 17, the operations further comprising displaying the heart-health index to a user.

20. The tangible machine-readable medium of claim 17, the operations further comprising generating a diagnosis by comparing the heart-health index against a specified threshold.
